(19) United States
(12) Patent Application Publication
(10) Pub. No.: US 2023/0364176 A1
(43) Pub. Date: Nov. 16, 2023

(54) ANTI-INFLAMMATORY FORMULATION-COATED TRANSDERMAL PATCH

(71) Applicants: Binoy K. Bordoloi, Bridgewater, NJ (US); KULWANT SINGH SAINI, GURUGRAM (IN)

(72) Inventors: Binoy K. Bordoloi, Bridgewater, NJ (US); KULWANT SINGH SAINI, GURUGRAM (IN)

(73) Assignee: Bordoloi Biotech LLC, Bridgewater, NJ (US)

(21) Appl. No.: 18/196,707

(22) Filed: May 12, 2023

Related U.S. Application Data
(60) Provisional application No. 63/342,063, filed on May 14, 2022.

(51) Int. Cl. A61K 36/906; A61K 36/899; A61K 36/758; A61K 9/70; A61K 9/06; A61K 47/14; A61K 47/32; A61K 47/10; A61K 47/18

(52) U.S. Cl. CPC A61K 36/906; A61K 36/899; A61K 36/758; A61K 9/7023; A61K 9/06; A61K 47/14; A61K 47/32; A61K 47/10; A61K 47/18

(57) ABSTRACT

The present invention relates to a transdermal patch mode of administration of topical synergistic anti-inflammatory formulation. The formulation comprises a blend 1 to 6% by weight of Cymbopogon citratus oil (CC oil); 0.5 to 6% by weight of Zanthoxylum armatum oil (ZA oil); 0.5 to 6% in weight by Hedychium spicatum oil (HS oil). The invention also provides a method of management of the pain and/or inflammation by the formulations coated on the transdermal patch of the invention.

### CROSS-REFERENCE TO RELATED APPLICATION

This application claims priority to U.S. Provisional Patent Application No. 63/342,063 titled “ANTI-INFLAMMATORY FORMULATION-COATED TRANSDERMAL PATCH” filed on May 14, 2022. The specification of the above referenced patent applications is incorporated herein by reference in its entirety.

### FIELD OF THE INVENTION

The present invention relates to transdermal patches coated with anti-inflammatory formulations, configured to exhibit significantly enhanced anti-inflammatory activity with reduced side effects.

### BACKGROUND

Arthritis is a disease due to the inflammation of connective tissue in joints, resulting in pain, swelling, stiffness and loss of function of the joints. The most common types of arthritis are osteoarthritis (OA) and rheumatoid arthritis (RA). Biologics based injections are the leading drugs for RA. For example, brands like Humira, Enbrel and Remicade accounted for 2015 sales exceeding S30 billion. Although effective, these drugs are expensive and are known to have certain side effects, which requires continuous medical attention of a physician. In general, these drugs block a protein called tumor necrosis factor (TNF), that's involved in inflammation. Therefore, these drugs are often called anti-TNF or TNF inhibitors. Similarly, Rumalaya forte of the Himalaya Drug Company, India is an oral dose medication that is reported to act as a TNF inhibitor. It is reported to be effective for both OA and RA, though it is not expected to be as effective as the biologic drugs.

OA is a chronic condition occurring more frequently as people get older and can break down joint's protective cartilage causing damage and pain. OA pain develops slowly and may require daily management for pain. While there is no known cure for OA, there are treatments that can help one relieve the pain in the joints. Physicians recommend the use of topical non-steroidal anti-inflammatory drugs (NSAIDs) for OA joint pain, at a lowest possible dose of medication for the shortest time to achieve pain relief. Voltaren Gel is one such available NSAID, used for the relief of joint pain of OA in the knee, ankles, feet, elbows, wrists and hands. It is applied on clean, dry skin that does not have any cuts, infections, or rashes. However, it is recommended that one should not use the gel along with any oral NSAIDs.

Doctors have long treated auto-immune diseases such as arthritis as a case of confused immune system. Here immune cells appear to decide that healthy tissue looks foreign and wage an attack. If the immune system is not actually confused but it is reacting to normal molecules that wound up in an abnormal place, the mechanism of action would be different. Padlock Therapeutics, USA thinks that the right way could be to block those errant molecules rather than blocking the immune system itself. This contrarian view of autoimmunity is based on evidence around a family of enzymes called protein arginine deiminases (PADs). Under normal conditions PADs live inside cells. But if they escape, immune cells start action, and a series of events begin to take place including what's known as protein citrullination, which is linked to RA. Padlock is designing small molecules to block this PAD activity.

Thus, techniques for managing OA and RA are based on man-made either biological large molecules or synthetic small molecules, however, there are medications also derived from plant extracts. These extracts are a mixture of several smaller molecules of varying chemistries. For example, Topricin is such a topical homeopathic cream to treat foot and ankle pain for neuropathy. Here the plant extract is a vasodilator, to treat the pain in the limbs relating to restricted blood flow to nerve endings and reduction in motor and sensory nerve conduction velocities.

Thus, a range of synthetic NSAIDs based formulations is available, which produce their own side effects based on the amount of active ingredient and duration of therapy. On the contrary, herbal formulations are also available which although are effective, but do not exhibit an effect that is higher or equivalent to the NSAIDs based topical formulations.

U.S. Pat. No. 7,871,647 discloses a composition for treating fibromyalgia pain consisting essentially of Aesculus hippocastanum extract, Arnica montana extract, L-arginine, Echinacea augustfolia extract, Rhus toxicondendron extract, Ruta graveoleus extract, graphites, Crotalus horridus extract, Heloderma horridum extract, Lachesis extract, Naja extract, isopropyl myristate, and capsaicin for homeopathic treatment.

U.S. Pat. No. 7,838,045 discloses an anti-inflammatory formulation of Barbarea verna seed containing phenethylisothiocyanate, which can be used for treatment of many inflammation-related conditions, including but not limited to rheumatoid and osteoarthritis, acute and chronic pains, lupus, irritable bowel disease, cancer and metabolic syndrome.

Mitoshi et. al. In International Journal of Molecular Medicine, 33, 1643-1651, 2014, published a study on the biological activity of twenty essential oils (EOs). The publication discloses Lemon Grass (Cymbopogon citratus oil or CC oil) to elicit strong anti-inflammatory and anti-allergic effects. The activity was determined by measuring the TNF-alpha in murine macrophage. Further, the principal component of CC oil was reported to be Citral.

Council for Scientific and Industrial Research and Northeast Institute of Science and Technology, India provides a topical ointment based on a blend of EOs of 2% Eucalyptus globulus oil, 2% Cinnamomum tamala oil, 2% Zanthoxylum armatum oil and 1% Hedychium spicatum oil for the treatment of OA and RA. The said gel based ointment was studied on both arthritic mice model and human clinical RA patients for effect on levels of cytokines.

D. Giri et al, in Journal of Medicinal Plants Research, Vol. 4(25), pp. 2773-2777, 2010 discloses the anti-inflammatory activity of Hedychium spicatum.

WO 2015000064 discloses a composition to treat pain and/or inflammation comprising beta-caryophyllene or a functionally equivalent derivative, analogue or pharmaceutically acceptable salt thereof, and eugenol or a functionally equivalent derivative, analogue or pharmaceutically acceptable salt thereof. It discloses beta-caryophyllen derived from Zanthoxylum armatum.

In addition, transdermal patches type of administration is used for reducing the amount of administered antibiotic or inflammatory formulations and related side effects. Although, the transdermal patches have certain advantages like minimized drug delivery, they have several shortcomings such as low skin impermeability and low loading capacity for drugs with high required dose.

However, there remains a need to develop new and alternative transdermal patches coated with anti-inflammatory formulations having enhanced synergistic anti-inflammatory effect. More specifically, there is a need to develop an improved transdermal patch coated with the anti-inflammatory formulations to provide an enhanced anti-inflammatory effect with a high benefit to low risk ratio. Also, there is a need for a transdermal patch mode of administration with high capacity and high anti-inflammatory activity.

### SUMMARY OF THE INVENTION

The present invention generally discloses transdermal patches for reducing pain in connective tissue of joints and muscles. Further, the present invention discloses transdermal patches coated/implemented/incorporated with anti-inflammatory formulation configured to exhibit significantly enhanced anti-inflammatory activity with reduced side effects.

In one aspect of the present invention there is provided a topical synergistic anti-inflammatory formulation coated on the transdermal patches comprising a blend of 1 to 6% by weight of Cymbopogon citratus oil (CC oil); 0.5 to 6% by weight of Zanthoxylum armatum oil (ZA oil); 0.5 to 6% by weight of Hedychium spicatum oil (HS oil).

In another aspect of the present invention there is provided a topical synergistic anti-inflammatory formulation coated on the transdermal patches comprising a blend of 1 to 6% by weight of Cymbopogon citratus oil (CC oil); 0.5 to 6% by weight of Zanthoxylum armatum oil (ZA oil); 0.5 to 6% by weight of Hedychium spicatum oil (HS oil) wherein the weight proportion of CC oil, ZA oil, and HS oil is 60%, 20% and 20% respectively.

In a further aspect of the invention there is provided a method of management of pain and/or inflammation in a mammal, said method comprising administering at the site of pain and/or inflammation a transdermal patch coated with a formulation comprising a blend of 1 to 6% by weight of Cymbopogon citratus oil (CC oil); 0.5 to 6% by weight of Zanthoxylum armatum oil (ZA oil); 0.5 to 6% by weight of Hedychium spicatum oil (HS oil).

In a yet another aspect of the invention there is provided a method of management of pain and/or inflammation in a mammal, said method comprising administering at the site of pain and/or inflammation a transdermal patch coated with a formulation comprising a blend of 1 to 6% by weight of Cymbopogon citratus oil (CC oil); 0.5 to 6% by weight of Zanthoxylum armatum oil (ZA oil); 0.5 to 6% by weight of Hedychium spicatum oil (HS oil); and wherein the weight proportion of CC oil, ZA oil, and HS oil is 60%, 20% and 20% respectively.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 shows a graphical representation of the method for collagen induced arthritis mice model in an embodiment of the present invention.

FIG. 2 shows a graphical representation of Cytokine TNF-alpha Level in various systems of EOs, namely (a) CC+ZA at 60% and 40%, (b) CC+HS at 60% and 40%, and (c) CC+ZA+HS at 60%, 20% and 20% respectively in an embodiment of the present invention.

FIG. 3 shows a graphical representation of Cytokine, IL-6 Level in various systems of EO's, namely (a) CC+ZA at 60% and 40%, (b) CC+HS at 60% and 40%, and (c) CC+ZA+HS at 60%, 20% and 20% respectively in an embodiment of the present invention.

FIG. 4 shows a graphical representation of Cytokine, IL-beta Level in various systems of EO's, namely (a) CC+ZA at 60% and 40%, (b) CC+HS at 60% and 40%, and (c) CC+ZA+HS at 60%, 20% and 20% respectively in an embodiment of the present invention.

### DETAILED DESCRIPTION OF EMBODIMENTS

The present invention is best understood by reference to the detailed figures and description set forth herein.

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

It is an object of the invention to overcome the drawback of the prior art.

It is another object of the invention to provide a transdermal patch coated/implemented/incorporated with a synergistic herbal formulation comprising Cymbopogon citratus oil [also known as Lemon Grass oil or CC oil], Zanthoxylum armatum oil (ZA oil), and Hydachium spicatum oil (HS oil).

It is another object of the invention to provide a transdermal patch coated with a topical formulation that has potential anti-inflammatory activity for symptoms such as frozen shoulder, stiff neck, backaches, musculoskeletal pains and rheumatoid arthritis, osteoarthritis, cervical and ankylosing spondylitis and sciatica.

It is yet another object of the invention to provide formulations-loaded transdermal patch as aforesaid that act without exerting toxic or side effects.

It is still another object of the invention to provide synergistic herbal formulations-coated transdermal patch that provides relief of minor aches and pain of muscle and joints in arthritis.

It is a further object of the invention to provide a method of administration of transdermal patch for delivering anti-inflammatory formulation configured to manage pain and/or inflammation in a mammal with minimal toxic or side effects.

The present invention relates to transdermal patches coated with the anti-inflammatory and analgesic formulation for management of inflammation disorder selected from the group consisting of rheumatoid arthritis, osteoarthritis, inflammatory bowel disease, Crohn's disease, ulcerative colitis, multiple sclerosis, psoriasis, and skin rashes. This includes frozen shoulder, stiff neck, backaches, musculoskeletal pains and rheumatoid arthritis, osteoarthritis, cervical and ankylosing spondylitis, sciatica, shoulder pain, back pain, lumbago, joint pain and swelling/pain after trauma from bruise and sprain.

The present invention provides the transdermal patch coated with a topical herbal formulation that provides anti-inflammatory and/or analgesic effect without producing any major side effects even on prolonged use. The invention discloses a blend of essential oils comprising Cymbopogon citratus (CC oil), Zanthoxylum armatum oil (ZA), and Hedychium spicatum oil (HS), which exhibit enhanced anti-inflammatory and analgesic effect compared to the known formulations having the individual essential oils. The herbal formulation can further include an essential oil obtained from basil, caraway, carrot seed, celery seed, chamomile, citronella, clary sage, clove, cumin, lemon, marjoram, nutmeg, orange, sage, sandalwood, spearmint, and/or thyme. In another instance, the blend of essential oils includes at least three oils selected from (A) Cymbopogon citratus (CC oil), (B) Zanthoxylum armatum oil (ZA), (C) Hedychium spicatum oil (HS), and (D) an essential oil obtained from basil, caraway, carrot seed, celery seed, chamomile, citronella, clary sage, clove, cumin, lemon, marjoram, nutmeg, orange, sage, sandalwood, spearmint, and/or thyme.

In the present invention, a transdermal patch coated with a topical anti-inflammatory and/or analgesic formulation comprising a blend of essential oils, namely, Cymbopogon citratus oil, Zanthoxylum armatum oil, and Hedychium spicatum oil showing synergistic effect is disclosed.

In an embodiment, the topical anti-inflammatory and/or analgesic formulation comprises a blend of essential oils at about 2 to 16% by weight; wherein said blend comprises: Cymbopogon citratus oil at about 1 to 6% by weight; Zanthoxylum armatum oil at about 0.5 to 6% by weight; and Hedychium spicatum oil at about 0.5 to 6% by weight.

The present inventors surprisingly found that upon combining the essential oils in a certain ratio, the formulation demonstrated synergistic activity while being non-toxic and safe. The said formulation comprising a blend of Cymbopogon citratus oil, Zanthoxylum armatum oil and Hedychium spicatum oil in a weight proportion of 60%, 20% and 20% respectively. Herein, the weight proportion provides the ratio of the essential oils; a weight proportion of 60%, 20%, 20%, provides a mass ratio of 3:1:1.

The formulation coated on the transdermal patch of the present invention further comprises menthol, a pure herbal compound, which produces a cooling sensation and has analgesic properties. Preferably, Menthol, (CAS No. 2216-51-5) of USP grade, water-white crystal and characteristic odor is used. The concentration of menthol in the formulation may vary from 1 to 16% by weight. Preferably, the concentration is 1.3% by weight. The transdermal patch is coated with a layer of the formulation of the present invention. The layer weighs about 200 gsm. In another embodiment, the transdermal patch comprises 11% w/w of medicated paste of the formulation. In one embodiment, the present invention discloses a method for treating pain. The method comprising a step of administering the transdermal patch coated with the synergistic anti-inflammatory formulation comprising the blend of essential oils.

In one embodiment, the transdermal patch comprises a protective liner to expose the adhesive side of the patch. Before application, the pain affected area is dried and one or more patches could be applied as directed by physician. Initially, the protective liner is removed from the patch to expose the adhesive side of the patch. Thereafter, the patch is placed over the pain affected area. The patch is a stretchable and waterproof patch and adheres to the skin of the patient. Further, the patch could be removed by peeling the patch gently or using warm water.

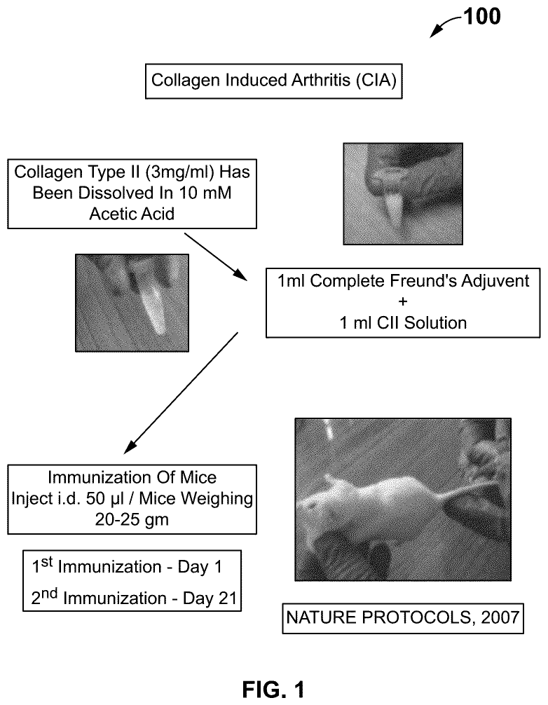

The formulation coated on the transdermal patch of the present invention is found to manage the symptoms of disease associated inflammation which are measured by the high levels of inflammatory cytokines in blood serum. Experiments were conducted for establishing the said effects. The increased level pro-inflammatory cytokines, for example, TNF-alpha, Interleukin-1beta (IL-1beta) and Interleukin-6 (IL-6), were measured quantitatively in blood serum in a collagen induced arthritic mice model 100 as shown in FIG. 1, and then compared with the effect after topical treatment with the present formulation, to establish the anti-inflammatory effect of the formulation.

The present inventors surprisingly observed that after topical treatment with the formulation of the present invention over a period of three weeks, the cytokine levels i.e. TNF-alpha, IL-6 and IL-1beta, showed a remarkable lowering, specifically to the extent of at least 50%.

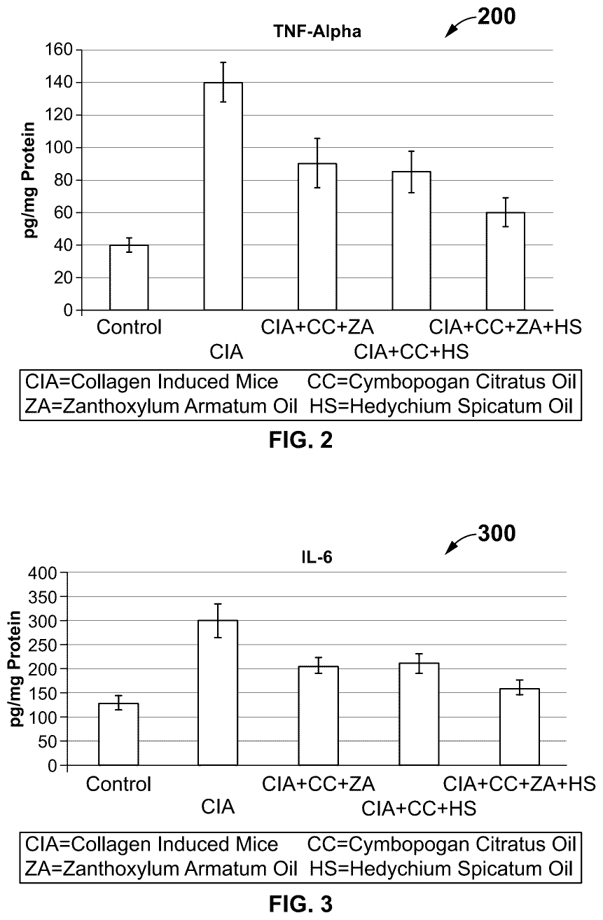

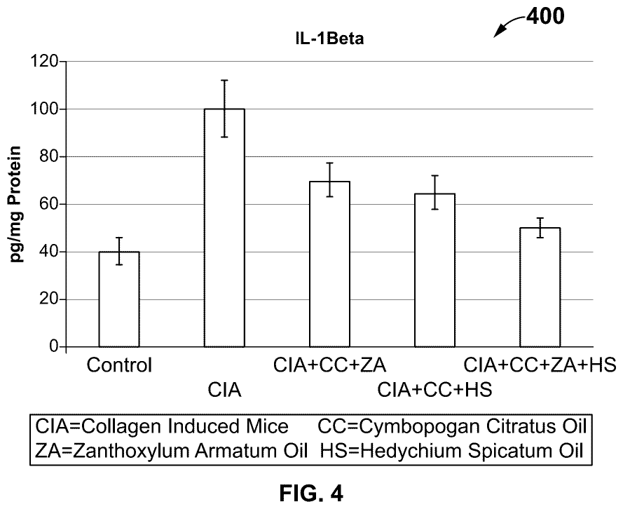

Referring to FIGS. 2, 3, and 4, a graphical representation (200, 300, and 400) of Cytokine TNF-alpha Level in various systems of EOs respectively, is illustrated. As observed in FIGS. 2, 3 and 4 there is remarkable decrease in the Cytokine Levels on Arthritic Mice; Control Healthy Mice Before Collagen Injection (Con), Arthritic Mice After Collagen Injection (CIA), Arthritic Mice After 21 Days of Treatment with Three Systems of EO's, namely (a) CC+ZA at 60% and 40%, (b) CC+HS at 60% and 40%, and (c) CC+ZA+HS at 60%, 20% and 20% respectively. Example FIGS. 2, 3 and 4 show that CC as well as CC oil with either ZA oil or HS oil as blends showed a decline in TNF-alpha, IL-6 and IL-1beta. Surprisingly, CC oil with a combination of ZA oil and HS oil, while maintaining the same total weight % of ZA and HS, showed consistently and significantly higher decline of the cytokine levels, displaying synergism in an unexpected manner Thus, also showing that the high anti-inflammatory effect is achieved while using less concentration of actives and thus reducing the toxic or side effects that may be produced on prolonged use.

As mentioned above the essential oils are formulated into various topical anti-inflammatory and/or analgesic formulations to incorporate into the transdermal patches.

In another embodiment, the formulation coated on the transdermal patch is provided as an oil-in-water type emulsion which includes fine particles of the essential oils. The fine particle of essential oil includes an effective amount of Cymbopogon citratus oil, Zanthoxylum armatum oil, and Hedychium spicatum oil and the Menthol crystals, in an aqueous medium and at least one emulsifier for dispersing the fine particles of the essential oils in the aqueous medium. The oil-in-water emulsion may include inactive oil(s) for better oil-water balance and emulsification. Examples of such oils include jojoba oil, coconut oil, almond oil, eucalyptus oil and avocado oil. The oil-in-water emulsion includes a base, preferably triethanolamine.

The formulation coated on the transdermal patch of the present invention may further comprise one or more of the conventional optional components known for use in topical anti-inflammatory and/or analgesic formulations, provided that the components used are compatible with the essential oils and menthol components of the formulation both physically and chemically. These optional components should generally not impair the stability, aesthetics or performance of the product. Concentrations of such optional components typically range from about 0.001% to about 25% by weight of the formulation.

Such conventional optional ingredients are well known to those skilled in the art and may be selected from the group comprising but not restricted to emulsifiers, co-emulsifiers, rheology modifiers, solubilizers, preservatives such as sodium benzoate, benzyl alcohol, methyl paraben and propyl paraben; thickeners and viscosity modifiers; perfumes; dyes; pH adjusting agents etc.

The formulation thus formed is incorporated into the transdermal patches for administrating topically and is indicated for the management of arthritis associated symptoms of pain and inflammation and related conditions including but not limited to rheumatoid arthritis, osteoarthritis, inflammatory bowel disease, Crohn's disease, ulcerative colitis, multiple sclerosis, psoriasis and skin rashes and other related disorders for reducing the production of pro-inflammatory cytokines.

The emulsifiers and co-emulsifiers are present in formulations in an amount of from 1 to 3%±1% by weight. These may preferably include Phoenoxol T, as emulsifier and Glyceryl stearate as co-emulsifier.

In an embodiment, the process for making the aqueous cream with the said blend of EOs i.e. wherein the weight proportion of CC oil, ZA oil, and HS oil is 60%, 20% and 20% respectively, includes the following steps: In phase-A, preservative and thickener are mixed together while heating. In phase-B, Emulsifier and Co-emulsifier are mixed together and heated, followed by addition of the EOs blend, so as to ensure minimum heating of the blend of oils. In phase-C, menthol is solubilized by mixing with solubiliziers and then warming. In phase-D, a base mixture is prepared to neutralize the pH from the added thickener to the required pH (We still keep it slightly acidic as the skin pH is acidic). Thus, four separate phases A, B, C, D are prepared. Then the phase B of blend of EOs in emulsifier is added to Phase A with preservative at 65-70° C. while rotating at 400 to 500 rpm. To the prepared mixture, phase D with base is added, while raising the rotation speed. Later, the formed mixture of Phase ABD is cooled and then phase C with solubilized menthol is added, while mixing. This is cooled and then mixed with deionized water in q.s. to obtain the cream.

In another aspect is provided a method of administrating transdermal patch type of delivery for managing pain and/or inflammation in a mammal. The method comprising administering at the site of pain and/or inflammation a formulation coated transdermal patch as described herein.

The terms “manage”, “management”, “treat”, “treating” and “treatment” are used broadly herein to denote methods that moderate, ameliorate, reverse the progression of, reduce the severity of, or prevent pain and/or inflammation. In this regard it is noted that because a wide range of inter-individual variability exists in the perception of pain, the perceived result of the present treatment may vary from mammal to mammal.

In one embodiment, the method includes administration of transdermal patch for delivering the formulation to a mammal in need of pain and/or inflammation treatment.

The formulation-coated transdermal patch is preferably applied onto the skin to promote absorption of the active ingredients. The formulation is administered at a dosage suitable to manage pain and/or inflammation. The formulation-loaded transdermal patch may be used for local and systemic delivery of the anti-inflammatories through skin for a sustained period of time. The duration may vary depending on the severity and other factors.

### EXAMPLES

The present invention is now demonstrated by way of illustrative non-limiting examples. Other variations as may be obvious to one skilled in the art may be made in compounds, formulations, and methods described herein without departing from the essential features of the invention. These and such others may be considered illustrative and non-restrictive to the scope of the present invention.

### Example 1

The 3 EO's and menthol was procured from Katyani Exports, Delhi, India. These included: Cymbopogon citratus oil, CC oil (CAS No. 8007-02-1), with a light yellow color and a strong, sharp odor of fresh-grassy lemon type; Zanthoxylum armatum oil, ZA oil (CAS No. 8015-91-6), with a yellow color; Hedychium spicatum oil, HS oil (no CAS No. was found to be assigned as yet), with a greenish yellow color; and Menthol, M (CAS No. 2216-51-5) of USP grade and water-white crystal with its characteristic odor.

These oils and crystal were used without further purification, though their infra-red (IR) and nuclear magnetic resonance (NMR) spectra, and liquid chromatography (LC) traces were collected and archived for batch-to-batch data collection.

Blends of the above stated EOs were made for experimentation at various ratios; such blends were then picked up with a micropipette for emulsification for making creams.

Various other ingredients were procured for making water based cream preparations; for example,
Deionized water, made at the lab premises via reverse osmosis method;
Preservatives: Gluconolactone and Sodium Benzoate, e.g. Geogard Ultra;
Thickener: Carbomer, e.g. Carbopol Ultrez-10;
Co-emulsifier: Glyceryl Stearate, e.g. Cutina GMSV;
Emulsifier: Cetearyl Alcohol and Steareth-30 and Ceteth-10, e.g. Phoenoxol T;
Solubilizer Propane Diol;
Solubilizer Glycerin;
Base (to assist in neutralization of the pH): Triethanolamine.

A cream formulation that is coated on the transdermal patch according to the present invention, comprising the combination of 3 Essential oils as mentioned in Table 1 is prepared and the formulation of the same is provided in Table 1.

TABLE 1
Composition of the present formulation.

| | |
|---|---|
| Ingredient | Wt % |

Phase-A

| | |
|---|---|
| Deionized Water | 76.2% |
| Gluconolactone and Sodium Benzoate - | 1.0% |
| preservatives | |
| Carbomer -thickener | 0.9% |

Phase-B

| | |
|---|---|
| Glyceryl Stearate -co-emulsifier | 2.0% |
| Cetearyl Alcohol and Ceteth-20 and | 3.0% |
| Steareth-10 Emulsifier | |
| Essential Oil - 1 (e.g. CC Oil) | 3.0% |
| Essential Oil - 2 (e.g. HS Oil) | 1.0% |
| Essential Oil - 3 (e.g. ZA Oil) | 1.0% |

Phase-C

| | |
|---|---|
| Propane Diol | 4.0% |
| Glycerin | 1.0% |
| Menthol | 1.0% |

Phase-D

| | |
|---|---|
| Deionized Water | 5.0% |
| Triethanoiamine - Base | 0.9% |
| Total | 100.0% |

### Example 2: Method for the Preparation of the Cream

The procedure for the preparation was as follows:
Step 1) Phase-A: Dispersing Carbopol in water, which is an acidic acrylic polymer. Then adding preservatives Gluconolactone and Sodium Benzoate, which are water-soluble. Heating the prepared mixture at 65 to 70° C. with mixing.
Step 2) Phase-B: Combining all the ingredients of Phase-B, and heating at 65 to 70° C. (This step has the EO's and the emulsifier mixed together the oils are added last when the temperature is reached so as to ensure minimum heating time for EOs.)
Step 3) Phase-C: Pre-mixing Phase-C in a separate vessel, and setting aside. (Menthol dissolves faster when warmed to 40° C.)
Step 4) Phase-D: Mixing Phase-D ingredients separately.
Step 5) Adding Phase-B to Phase-A at 65 to 70° C. with mixing at 400 to 500 rpm. Step 6) Adding Phase-D to the blend of A and B, mixing and raising the speed to 900 rpm, as the emulsion thickens.
Step 7) Allowing the blend of ABD to cool to below 40° C. while mixing, and then adding Phase-C to ABD, and mixing.
Step 8) Cooling down the above blend of ABCD to room temperature with mixing and QS to 100 using DI-water. (QS means that you add DI-water such that the final weight is 100 g; it is assumed that the water phase evaporates partially during cream preparation.)

### Example 3

The various properties of the above prepared cream of example 2 were studied.

A pH meter and a Brookfield viscometer were used for measuring pH and viscosity, respectively, of the cream prepared. The above resulted in a white cream of pH 5.5 and a viscosity of 180,000 cSt at 21° C.

Stability of the cream produced was evaluated after aging at 50° C. for four weeks in screw-cap glass containers. It gave a stable viscosity in the range of 150,000 to 200,000 cSt and a pH of about 5.0 to 5.1 at 21° C.

It was also observed that the cream was stable throughout five cycles of freeze-thaw, and it did not show changes in appearance or any sign of separation.

It is concluded that although it is known that CC oil shows a reduction in inflammatory cytokine levels, or that ZA oil and HS oil display a reduction in inflammatory cytokine levels, the results observed in FIGS. 2, 3, and 4 for the blend of CC oil, ZA oil and HS oil of the present invention composition were found to be unique, unexpected, and never reported earlier. While the weight proportions of CC oil and ZA oil, or CC oil and HS oil were held constant at 60% and 40% for the first oil and second oil respectively, it was clearly observed that the blend of CC oil, ZA oil and HS oil as comprised in the present composition demonstrated significant synergy in reducing the inflammatory cytokines. This was a consistent behavior in each of the cases of TNF-alpha (TNF-α), Interleukin-1beta (IL-1b) and Interleukin-6 (IL-6). Therefore, the blend CC oil, ZA oil and HS oil as comprised in the present composition is considered to have surprisingly beneficial anti-arthritic effects. Further, the transdermal patch as a delivery system for anti-inflammatory formulation may prevent inflammations and may aid in decreasing unnecessary administration of other formulations.

Preferred embodiments of this invention are described herein, including the best mode known to the inventors for carrying out the invention. It should be understood that the illustrated embodiments are exemplary only and should not be taken as limiting the scope of the invention.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A transdermal patch for pain management, comprising:
a synergistic anti-inflammatory formulation comprising a blend of essential oils, wherein the blend includes at least three essential oils selected from (a) Cymbopogon citratus oil (CC oil), (b) Zanthoxylum armatum oil (ZA oil), (C) Hedychium spicatum oil (HS oil), and (d) a fourth essential oil obtained from basil, caraway, carrot seed, celery seed, chamomile, citronella, clary sage, clove, cumin, lemon, marjoram, nutmeg, orange, sage, sandalwood, spearmint, and/or thyme.

2. The transdermal patch of claim 1, wherein the blend includes:
about 1 to about 6% by weight of the CC oil;
about 0.5 to about 6% by weight of the ZA oil, and
about 0.5 to about 6% by weight the HS oil.

3. The transdermal patch of claim 1, wherein the weight proportion of the CC oil, the ZA oil, the HS oil in the blend of essential oils is about 60%, about 20%, and about 20%, respectively.

4. The transdermal patch of claim 1, wherein the formulation further comprises menthol in an amount from about 1 to about 16% by weight of the formulation.

5. The transdermal patch of claim 1, wherein the formulation is in the form of emulsion, and wherein the emulsion comprises an emulsifier.

6. A method for treating pain, comprising: administering a transdermal patch to a patient in need thereof, the transdermal patch coated with a synergistic anti-inflammatory formulation comprising a blend of essential oils, wherein the blend includes at least three essential oils selected from (a) Cymbopogon citratus oil (CC oil), (b) Zanthoxylum armatum oil (ZA oil), (C) Hedychium spicatum oil (HS oil), and (d) a fourth essential oil obtained from basil, caraway, carrot seed, celery seed, chamomile, citronella, clary sage, clove, cumin, lemon, marjoram, nutmeg, orange, sage, sandalwood, spearmint, and/or thyme.

7. The method of claim 6, wherein the blend includes:
about 1 to about 6% by weight of the CC oil;
about 0.5 to about 6% by weight of the ZA oil, and
about 0.5 to about 6% by weight the HS oil.

8. The method of claim 6, wherein the weight proportion of the CC oil, the ZA oil, the HS oil in the blend of essential oils is about 60%, about 20%, and about 20%, respectively.

9. The method of claim 6, wherein the formulation further comprises menthol in an amount from about 1 to about 16% by weight of the formulation.

10. The method of claim 6, wherein the formulation is in the form of emulsion, and wherein the emulsion comprises an emulsifier.
